(19) United States
(12) Patent Application Publication
(10) Pub. No.: US 2025/0169756 A1
(43) Pub. Date: May 29, 2025

(54) Over-Shoe Contact Sensor for Gait Measurement in Physical Therapy

(71) Applicant: NextStep Robotics, Inc., Baltimore, MD (US)

(72) Inventors: Bradley Hennessie, Columbia, MD (US); Anindo Roy, Baltimore, MD (US); Nicholas Rabuck, Severn, MD (US); Douglas Summerlin, Mount Airy, MD (US)

(21) Appl. No.: 18/954,665

(22) Filed: Nov. 21, 2024

Related U.S. Application Data
(60) Provisional application No. 63/603,858, filed on Nov. 29, 2023.

(51) Int. Cl. A61B 5/00; A61B 5/103

(52) U.S. Cl. CPC A61B 5/6807; A61B 5/1038

(57) ABSTRACT

An over-shoe contact sensor device enabling a novel method and a portable embodiment for reliably and accurately detecting multiple gait events with several key attributes particularly advantageous in physical therapy applications such as compact size, quick donning and doffing, and durability to wear and tear. The device is a cost-effective alternative to existing anchored motion tracking systems when utilized for the same purpose. The device comprises a plurality of sensors for measuring gait events and under-the-foot forces and processing data collected from the sensors to provide a useful output for physical therapists and the like to determine the gait of the wearer.

### CROSS-REFERENCE TO RELATED APPLICATIONS

This application claims the benefit of priority of U.S. Provisional Patent App. Ser. No. 63/603,858, the contents of which are incorporated herein by reference in their entirety.

### BACKGROUND OF THE INVENTION

Gait tracking is an essential aspect of physical therapy for assessing the progress of patients recovering from injuries or surgeries affecting their walking patterns. In particular, accurate and reliable acquisition of gait events is a prerequisite for integration with robotic lower limb rehabilitation systems that rely on providing timed support to the patient. Additionally, the measurement of under-the-foot forces during walking is crucial for detecting gait abnormalities, which can indicate underlying musculoskeletal or neurological issues. Existing methods involve external instrumentation such as anchored infrared-based motion capture systems or portable inertial measurement units (IMUs), etc. However, these systems have limitations such as prohibitively high cost, large physical footprints (especially for anchored instrumentation), or lack of open architecture that precludes integration with custom hardware or software. There exists a need for low cost, compact, quick to don and doff, and durable solutions to provide reliable and accurate timing of gait events and force distribution for integration into custom robotic rehabilitation hardware and software.

Alternative options for gait tracking include external sensors (pressure sensing mats, camera arrays, infrared sensor arrays, etc.), insole pressure sensors, or acceleration sensors. While these methods can provide some information about gait, they often lack the ability to accurately measure under-the-foot forces which is essential for identifying subtle gait abnormalities. It is an object of the present invention to track the timing of key gait events and measure under-the-foot forces using an over-shoe contact sensor, which reliably and accurately provides this information while being compact, quick to don and doff, and durable.

### BRIEF SUMMARY OF THE INVENTION

The invention according to the present disclosure comprises an over-shoe contact sensor, which is an innovative solution for gait event detection and tracking. The underlying methods and embodiments are designed to overcome the limitations of existing methods. The sensor consists of five metal sheets adhered on opposite sides of a foam sheet consisting of holes in areas of interest (i.e., those implicated in key events of the gait cycle) where pads of thinner, less compressible conductive rubber are placed. Each conductive rubber pad functions as a pressure-sensitive resistor, with its resistance decreasing as the pressure on it increases. When the sensor is compressed, the foil sheets move towards each other until they contact the conductive rubber, creating an electrical short that is measured to determine if the sensor is compressed in that given area. The magnitude of the current flowing through the circuit indicates the overall force applied to the sensor, and by analyzing the current through each individual pad, the distribution of under-the-foot forces across the sensor can be determined. The sensor is enclosed in rubber to accommodate regular wear and tear and embedded in an adjustable overshoe that accommodates a range of shoe sizes for both men and women.

### BRIEF DESCRIPTION OF THE DRAWINGS

FIG. 1 illustrates an exemplary design of a crampon according to the present disclosure.

FIG. 2 illustrates a subsection of an exemplary design of a crampon according to the present disclosure.

FIG. 3 illustrates an exemplary design of a sensor wiring layout according to the present disclosure.

FIG. 4 includes photographs of an exemplary embodiment of the present invention.

FIG. 5 illustrates a subsection of a sensor cross-section of an exemplary embodiment of the present invention.

### DETAILED DESCRIPTION OF THE INVENTION

The invention described herein comprises an over-shoe contact sensor that is a compact and durable device designed for dynamic gait event detection in robot-aided physical therapy and other applications that utilize gait event signals in physical human-robot interaction control and gait analysis in the physical therapy setting.

In certain embodiments, a device according to the present disclosure comprises a minimum of two foil (metal) sheets, which ideally are highly conductive material sheets made of brass and adhered on opposite sides of a foam sheet made of polyurethane. The device further comprise a minimum of three pads made of conductive rubber, such as silicon elastomer with a conductive nickel-graphite powder filler, strategically placed in holes that represent areas implicated in key events of the gait cycle. Each conductive rubber pad is configured to function as a pressure-sensitive resistor, where the resistance of the pad decreases as the pressure applied to it increases. An electrical circuit is included that comprise a force-invoked, momentary push-button switch that creates an electrical short if a connected sensor is compressed in a given region. The total current flowing through the circuit is a measure of the overall force applied to the sensor. By analyzing the current flow through individual pads, the under-the-foot forces across the sensor can be determined. The device may further comprise a rubber enclosure around the sensor to protect it from damage. The sensor is encased in a rubber shell which stabilizes the internal electrical components, protects the electronics from external environmental factors, and firmly anchors the sensor to an adjustable overshoe.

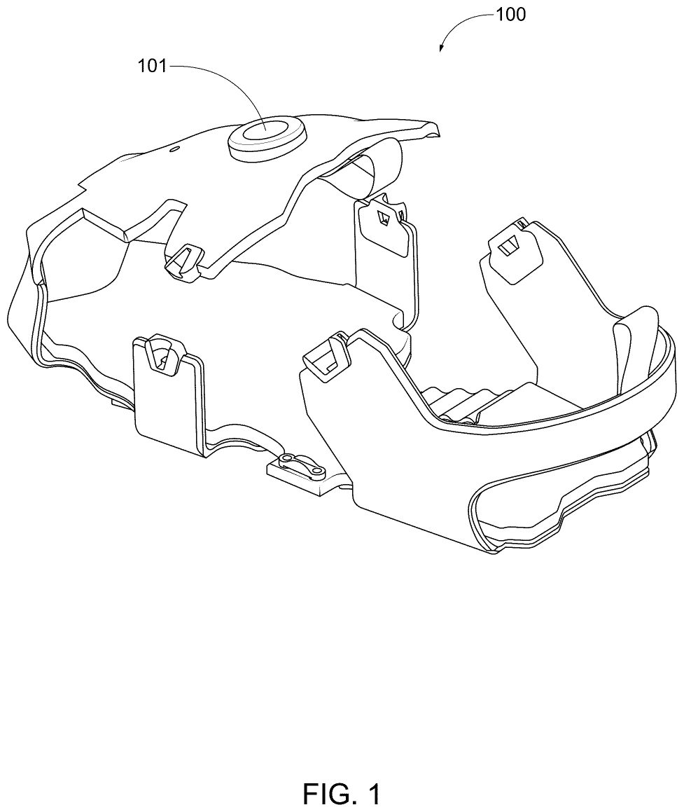

FIG. 1 illustrates adjustable overshoe 100 as described herein in the form of a crampon consisting of flexible rubber that conforms to the profile of the wearer's foot via an adjustable dial mechanism 101, as will be familiar to one of ordinary skill in the art. The over-shoe contact sensor 100 reliably and accurately captures gait events, while maintaining several key attributes required for integration into clinical workflow for rehabilitation applications including but not limited to low manufacturing cost, quick donning times, and quick doffing times. Certain embodiments may be available for most shoe sizes (small, medium, large) in males (men's US sizes 7-14) and females (women's US sizes 8-12). These and other features will be appreciable to those of ordinary skill in the art.

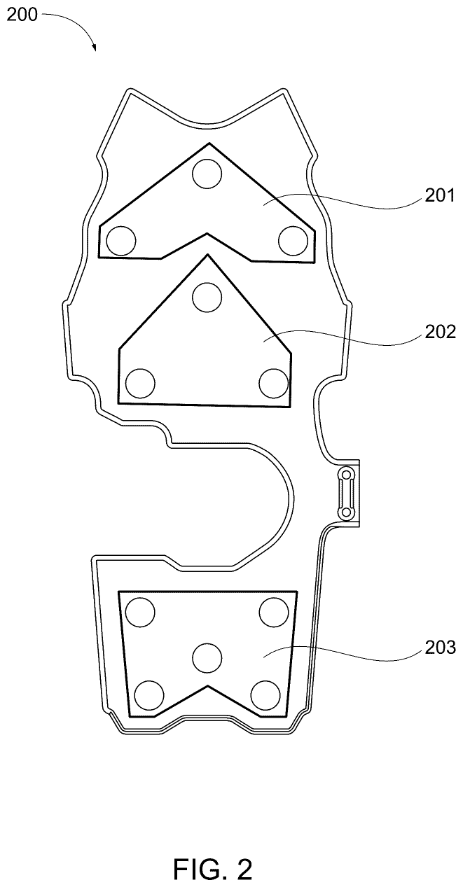

FIG. 2 illustrates the bottom of a sensor crampon 200 having sensor assemblies according to the present disclosure is designed to activate at a particular force threshold. This threshold is sensitive enough to ensure reliable activation of the sensor crampon 200 during walking, while not being too sensitive to the point that it erroneously activates from abnormal gait, variable shoe size, and external shoe geometry. The activation range varies based on the size of the crampon (small, medium, and large). Across all sizes of sensor crampon, the activation force ranges from 40 to 100 pound-feet. Table 1 below provides details.

This force data was gathered using a force gauge to manually apply pressure until a footswitch sensor tool indicated that the region had been activated (i.e., shorted to ground). A custom jig was made to position the sensor crampon while the force was applied. Refer to the pictures below for details.

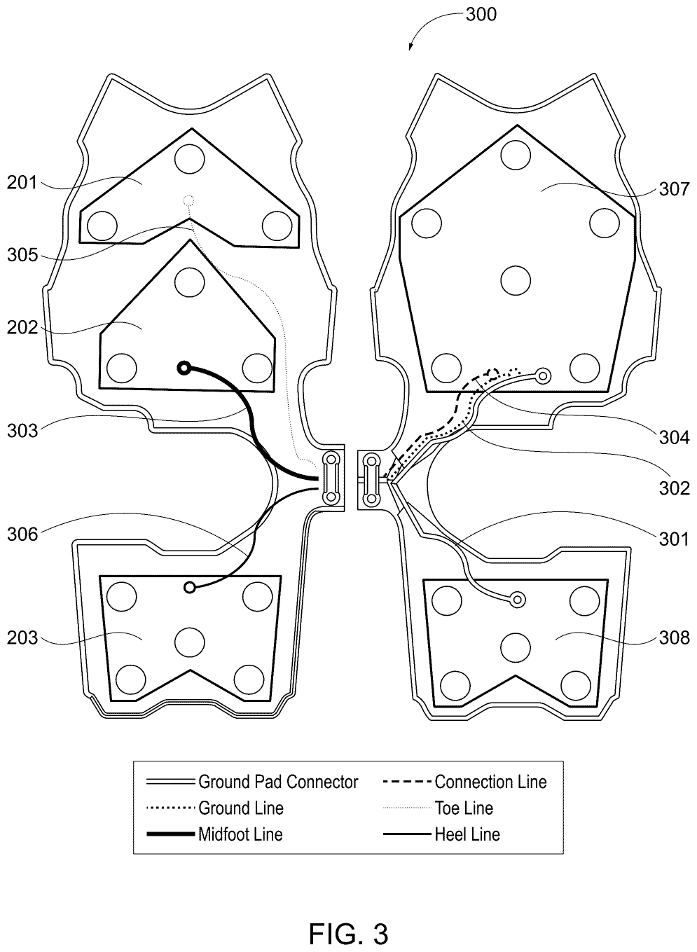

As illustrated in FIG. 3, a sensor assembly 300 may comprise a ground pad protector 301, ground line 302, midfoot line 303, connection line 304, toe line 305 and heel line 306. Sensors may be arranged as illustrated in FIG. 2 (201, 202, 203) or FIG. 3 (307, 308). The overall design is configured to process sensor data from each region of the foot to determine overall gait.

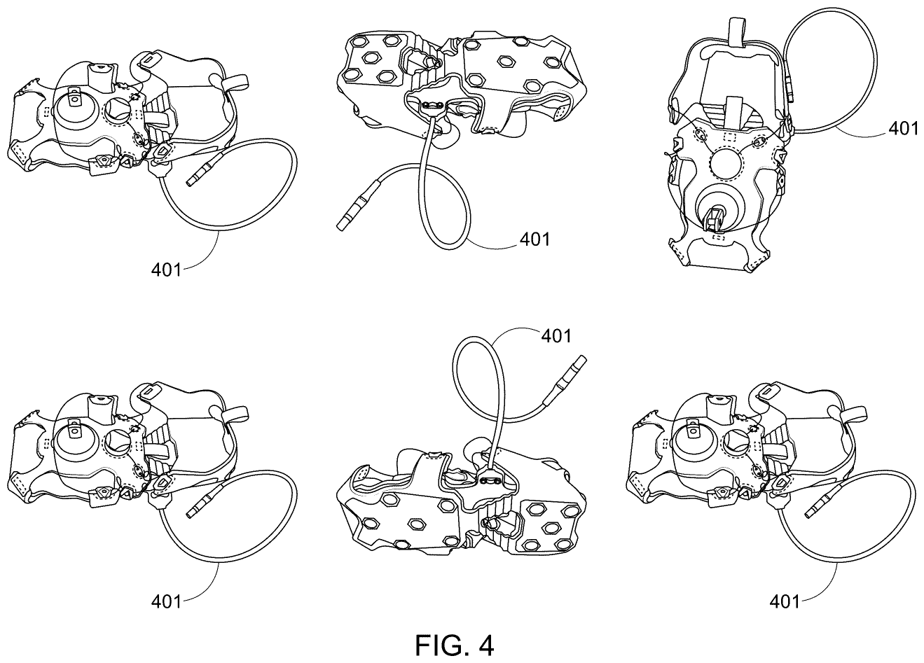

Information may be transmitted electrically by a connected wire 401 as illustrated in FIG. 4 using a variety of selected connection points. Collected information may be transmitted via Bluetooth, WiFi or other means to a processor that provides a readout of the information.

TABLE 1
Activation Force (in pound-feet)

| | Size of Crampon | | |
|---|---|---|---|
| Sensor Region Acceptability | Small | Medium | Large |
| Toe Under-Sensitivity Criteria | 90 | 60 | 60 |
| Midfoot Under-Sensitivity Criteria | 85 | 70 | 70 |
| Heel Under-Sensitivity Criteria | 100 | 85 | 85 |
| Toe Over-Sensitivity Criteria | 50 | 40 | 40 |
| Midfoot Over-Sensitivity Criteria | 50 | 40 | 40 |
| Heel Over-Sensitivity Criteria | 60 | 40 | 40 |
| Average | 73 | 56 | 56 |
| Max | 100 | 85 | 85 |
| Min | 50 | 40 | 40 |
| | Max | Min | |
| Overall Range Across Sizes (lbf) | 100 | 40 | |

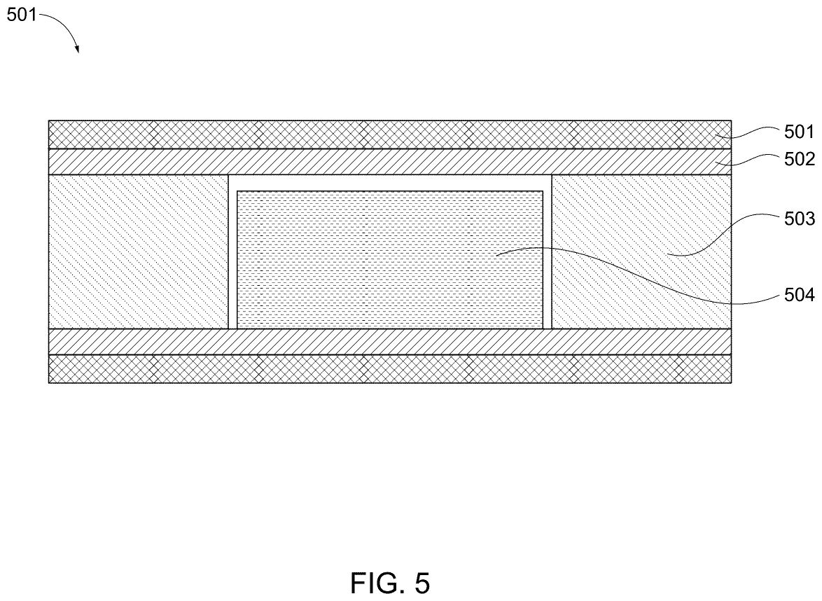

As shown in FIG. 5 in cross-section, a sensor 500 according to the present disclosure may comprise two rubber casings 501, two foil sheets 502, a foam component 503 that compresses, and a conductive rubber pad 504. The two foil sheets 502 are adhered on opposite sides of the foam component 503. In the foam component 503, holes are made in areas of interest and conductive rubber added within the holes. This causes the two foil sheets 502 to short circuit, and the electrical short between the two foil sheets 502 is measured to determine if the sensor is compressed in a given area. This leads to a rugged design that can embed in an adjustable overshoe to ensure proper placement on the underside of the overshoe.

TABLE 2
Example Detectable Gait Events

| Event | Toe | Midfoot | Heel | Event Name |
|---|---|---|---|---|
| 0 | 0 | 0 | 0 | Swing-Phase |
| 1 | 0 | 1 | 1 | Early Stance Phase |
| 2 | 1 | 1 | 0 | Late stance Phase |
| 3 | 1 | 0 | 0 | Toe-off event |
| 4 | 0 | 0 | 1 | Heel-first event |
| 5 | 1 | 1 | 1 | Foot-flat event(mid stance) |

One of ordinary skill in the art will recognize the importance of tracking the timing of key gait events on the leg to which the force is applied. The toe, midfoot and heel are read as a matrix of ones and zeroes based on whether they are compressed or not, respectively. In this way, gait events such as swing phase, early stance phase, late stance phase, toe-off event, heel-first event and foot-flat event (mid stance) may be computed. Table 2 illustrates the basics of gait computation according to the present disclosure.

What is claimed is:

1. A method for capturing gait events and under-the-foot forces during level or incline walking, the method comprising:
donning an over-shoe contact sensor configured to capture said gait events and under-the-foot forces by detecting compression during said gait events; and
walking on a level or inclined surface as initial contact, toe-off, and full weight bearing.

2. The method of claim 1, wherein said gait events are selected from the group consisting of initial contact, toe-off, full weight-bearing and combinations thereof.

3. A method for measuring under-the-foot forces during gait, comprising:
using the apparatus of claim 1 in which a measure of total current in the over-shoe contact sensors is used to generate force distribution data during walking.

4. An adjustable over-shoe contact sensor apparatus comprising:
at least two metal foil sheets;
at least three conductive rubber pads;
an electrical circuit comprising a force-invoked, momentary push-button switch that creates an electrical short if a connected sensor is compressed in a selected region;
a rubber enclosure around said sensor; and
an adjustable over-shoe.

5. The adjustable over-shoe contact sensor apparatus of claim 4, wherein the at least two metal foil sheets are made of brass and adhered on opposite sides of a foam sheet made of polyurethane.

6. The adjustable over-shoe contact sensor apparatus of claim 4, further comprising at least three pads made of conductive rubber.

7. The adjustable over-shoe contact sensor apparatus of claim 6, wherein the conductive rubber comprises a silicon elastomer with a conductive nickel-graphite powder filler located in holes that represent areas implicated in key events of the gait cycle, and wherein the conductive rubber is configured to measure under-the-foot forces across the area of each hole.

8. The adjustable over-shoe contact sensor apparatus of claim 4, further comprising an electrical circuit having a force-invoked, momentary push-button switch that creates an electrical short if a connected sensor is compressed in a selected region.

9. The adjustable over-shoe contact sensor apparatus of claim 4, further comprising a rubber enclosure around the sensor to protect it from damage.

10. The adjustable over-shoe contact sensor apparatus of claim 4, further comprising a crampon consisting of flexible rubber that conforms to the profile of the wearer's foot via an adjustable dial mechanism.
